(19) United States
(12) Patent Application Publication
(10) Pub. No.: US 2018/0110708 A1
(43) Pub. Date: Apr. 26, 2018

(54) CLEANING/CONDITIONING SHAMPOO

(71) Applicant: Kao USA, Inc., Cincinnati, OH (US)

(72) Inventor: Adam SCHROTT, Cincinnati, OH (US)

(21) Appl. No.: 15/334,348

(22) Filed: Oct. 26, 2016

(51) Int. Cl. A61K 8/42; A61K 8/92; A61Q 5/02; A61Q 5/12; A61K 8/36; A61K 8/34

(52) U.S. Cl. CPC A61K 8/42; A61K 8/922; A61K 8/342; A61Q 5/12; A61K 8/361; A61Q 5/02

(57) ABSTRACT

The present application discloses hair care compositions which both clean and condition hair. The compositions comprise a defined amidoamine material, a long chain fatty alcohol, a nonionic amide surfactant as defined herein, and water as the solvent. The composition may additionally include an oil which is liquid at room temperature. The method of cleaning and conditioning hair using the defined composition is also disclosed.

### BACKGROUND

The present invention relates to a hair shampoo composition which both effectively cleans and conditions hair during a simple and quick shampooing operation.

Hair care is a very important part of most people's personal grooming routine. Hair is cleaned by shampooing and then conditioned in order to improve its visual characteristics and combability.

Shampoos are well-known products which are typically applied to wet hair, worked into the hair and then rinsed out, thereby providing clean hair.

Hair conditioners are utilized frequently to improve the texture and appearance of human hair. These formulations may confer specific benefits that are broadly defined as hair manageability. More specifically, hair manageability may be thought of as a combination of benefits such as reduction and/or removal of static charge, detangling, strengthening, split end removal, shine improvement, thickening, fragrance delivery, moisturization, lubrication, and other properties that generally result in the perception of repair of damaged hair and of enhanced beautification.

Frequently, the shampooing and conditioning of hair are carried out in two separate steps. But this can be time consuming. Compositions have been developed that provide conditioning benefits simultaneously with cleaning, and are known in the field as “two-in-one shampoos”. Existing two-in-one shampoos fall short of desired expectations in several different ways. Many are simply hair conditioner products which have been renamed and claim to provide cleansing in addition to hair conditioning. Such products tend to be devoid of cleansing/detersive surfactants and, while they provide conditioning, they typically do not provide a strong level of cleaning. This can lead to buildup on the hair and result in greasiness and weigh-down to the user. Some products address this shortcoming by adding an anionic or nonionic surfactant to the conditioner product. While these products add a more traditional type anionic surfactant to a conditioner base, both cleansing and conditioning become compromised as a result of the interaction of the anionic surfactant and the cationic conditioning material. Not enough cleansing is provided and sebum remains on the scalp while the conditioning effect is not strong enough and tangles still result.

U.S. Published Patent Application 2016/0143827, Castan Barbaran et al, published May 26, 2016, relates to a hair conditioner composition, which is solid at room temperature, and which comprises one or more quaternary ester ammonium compounds (a), one or more amidoamine compounds (b), and one or more fatty alcohols (c); wherein the weight ratio between components (a) and (b) fall within the range of 1:5 to 5:1.

European Patent Application EP 1 779 838 A1, Hoffmann et al, published May 2, 2007, describes a hair conditioning composition which provides the hair with enhanced body and volume, especially for fine hair, which comprises polyquaternium-37, at least one fatty alcohol, and at least one mono-long chain quaternary ammonium cationic surfactant or nonionic surfactant.

The present invention addresses these issues and provides a two-in-one shampoo/conditioner product which both effectively cleans and conditions the hair in a single operation, when used.

### SUMMARY

The present invention relates to a two-in-one shampoo/conditioner hair composition which comprises:

(a) an amidoamine with the structure:

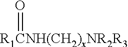

wherein R₁ is an alkyl radical with n carbon atoms (n=8-24), benzyl, and mixtures thereof; wherein R₂ is —H, —CH₃, —CH₂OH, —CH₂CH₃, —CH₂CH₂OH, —CH₂CH₂CH₂OH, —CH₂(CHOH)₄CH₂OH, -benzyl, and mixtures thereof; wherein R₃ is —H, —CH₃, —CH₂OH, —CH₂CH₃, —CH₂CH₂OH, —CH₂CH₂CH₂OH, —CH₂ (CHOH)₄CH₂OH, -benzyl, and mixtures thereof; and X=1 to 3;

(b) a fatty alcohol having 12 to 24 carbon atoms;

(c) an amide surface active agent having the structure:

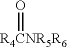

wherein R₄ is lauric acid, coconut acid, palmitic acid, myristic acid, behenic acid, babassu acid, isostearic acid, stearic acid, corn fatty acid, soy fatty acid, soy fatty acid, shea butter fatty acid, caprylic acid, capric acid, and mixtures thereof;

wherein R₅ is —CH₂OH, —CH₂CH₂OH, —CH₂CH₂CH₂OH, —CH₂(CHOH)₄CH₂OH, -benzyl, and mixtures thereof; and

wherein R₆ is —H, —CH₃, —CH₂OH, —CH₂CH₃, —CH₂CH₂OH, —CH₂CH₂CH₂OH, —CH₂(CHOH)₄CH₂OH, -benzyl, and mixtures thereof; and

(d) from about 5% to about 95% water;

wherein the composition comprises greater than about 2% of the total of components (a) and (b).

The composition may additionally include an oil material which is liquid at room temperature (25° C.).

Finally, the present invention incorporates a method of cleaning and conditioning hair utilizing the shampoo/conditioner compositions defined herein.

All ratios and percentages set forth in this application are “by weight” unless otherwise specified. Further, any patents, patent applications or other publications referenced in this application are incorporated by reference herein.

### DETAILED DESCRIPTION

The compositions of the present invention include a specifically defined amidoamine material, a long chain fatty alcohol, a specifically defined amide nonionic surfactant, and an aqueous solvent. This invention approaches combined cleaning and hair conditioning in a unique way. Specifically, detersive nonionic surfactants are combined with conditioning nonionic surfactants to provide a balance of cleaning with detangling and anti-static effects. This balance, and improved sebum cleansability, can further be enhanced with the addition of an oil which remains liquid at room temperature. This effectively increases the fluidity and therefore the removability of fatty sebum residues on the scalp.

Amidoamine Compounds

The present invention comprises one or more amidoamine hair care compounds, wherein the one or more amidoamine compounds preferably comprise at least an amidoamine compound of the following formula or even more preferably the one or more amidoamine compounds are all represented by this formula:

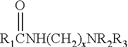

wherein R₁ is selected from an alkyl radical with n carbon atoms (n=8 to 24), benzyl, and mixtures thereof;

wherein R₂ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof;

wherein R₃ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof;

and X=1 to 3.

The amidoamine component (a) is utilized at from about 0.5% to about 5%, for example from about 0.75% to about 3.75%, of the shampoo/conditioner compositions of the present invention.

In one embodiment of the present invention, the one or more amidoamine compounds are represented by the formula above wherein R₁ represents a linear or branched, saturated or unsaturated alkyl chain group having from 8 to 24 carbon atoms, for example, from 12 to 24 carbon atoms; and R₂ and R₃ each independently represent a methyl group.

In another embodiment of the present invention, the amidoamine compound is selected from the group consisting of stearamidopropyl dimethylamine, babassu amidopropyl dimethylamine, and behenamidopropyl dimethylamine, as well as mixtures of those materials.

The amidoamine compounds used herein can be obtained by the reaction of a fatty acid (natural origin or technical mixture of fatty acids) and dimethylaminopropylamine.

Examples of commercially available amidoamines are those corresponding to the tradenames AMIDET APA-18 (stearamidopropyl dimethylamine), and AMIDET APA-22 (behenamidopropyl dimethylamine), both of them marketed by Kao Chemicals Europe.

Another group of amidoamine materials useful herein are those which comprise a mixture of stearamidopropyl dimethylamine and behenamidopropyl dimethylamine, described in U.S. Published Patent Application 2014/0328784, Burgo et al, published Nov. 6, 2014, incorporated herein by reference.

Fatty Alcohols

The compositions of the present invention also include from about 2% to about 15%, such as from about 4% to about 13%, of at least one fatty alcohol. The fatty alcohols are linear or branched, saturated or unsaturated with from 10 to 24, for example, 12 to 22, carbon atoms in their alkyl chains. Non-limiting examples of such materials include myristyl alcohol, cetyl alcohol, stearyl alcohol, cetearyl alcohol, palmityl alcohol, oleyl alcohol, lauryl alcohol, coconut alcohol, palm alcohol, behenyl alcohol, arachidyl alcohol, and mixtures of those materials. Exemplary embodiments include cetearyl alcohol, cetyl alcohol, myristyl alcohol and stearyl alcohol, as well as mixtures of those materials.

The compositions of the present invention should include at least about 2% of the combination of components (a) and (b). In some embodiments, the weight ratio of components (a):(b) is from about 1.3:1 to about 1:7.

Amide Nonionic Surfactants

The compositions of the present invention also include from about 1% to about 7%, such as from about 1% to about 5%, of an amide nonionic surfactant having the formula:

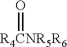

wherein R₄ is selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof;

wherein R₆ is selected from —CH₂OH, —CH₂CH₂OH, —CH₂CH₂CH₂OH, —CH₂(CHOH)₄CH₂OH, -benzyl, and mixtures thereof;

wherein R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

Examples of materials for use in the present compositions include cocamide monoethanolamine, cocamide diethanolamine; lauramide monoisopropylamide (MIPA), soyamide diisopropylamine (DIPA), and capryloyl/caproyl methyl glucamide, as well as mixtures of those materials.

In some embodiments, the weight ratio of components (a):(c) is from about 4:1 to about 1:4.

Solvent

The present invention utilized an aqueous solvent. As a solvent, the compositions of the present invention include from about 5% to about 95% of water. The solvent can also be a water/alcohol (e.g., ethanol) mixture.

The materials utilized in the present compositions are selected, consistent with the definitions given herein, such that they are compatible with the remainder of the ingredients in the compositions, as well as suitable for application to the hair and skin.

The compositions of the present invention generally have a pH range of from about 3 to about 6 (neat). The compositions may include a pH control agent, such as citric acid, lactic acid, benzoic acid, gluconic acid, metal hydroxides, triethanolamine, monoethanolamine, and mixtures of those materials, in order to adjust the composition pH within the desired range.

Optional Components

The compositions of the present invention may include an oil which is liquid at room temperature (about 25° C.). When used, this oil constitutes from about 0.5% to about 10%, such as from about 0.5% to about 5% of include mineral oil, abyssinian oil, sunflower seed oil, and canola oil, as well as mixtures of those materials. In one embodiment, the oil materials include from about 50% to about 70% by weight of components having a chain length of greater than or equal to C₂₂. An example of such preferred material is crambe abyssinica seed oil. This oily material can act to improve the conditioning and removal of sebum provided by the compositions of the present invention, as well as improve the aesthetics of the final composition.

Emulsifiers, generally nonionic, may also be included in the compositions of the present invention in order to compatibilize the oily and aqueous components found in the compositions. Examples of nonionic emulsifiers which can be used in the present invention include sorbitan fatty acid esters (such as sorbitan monoleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, and diglycerol sorbitan, tetra-2-ethylhexylate; glycerol or polyglycerol fatty acid esters (such as glycerol mono-cotton seed oil fatty acid ester, glycerol monoerucate, glycerol sesquioleate, glycerol monostearate, glycerol-α, α′-oleate pyroglutamate, and glycerol monostearate malate); propylene glycol fatty acid esters (such as propylene glycol monostearate); hardened castor oil derivatives; and glycerol alkyl ethers. Exemplary nonionic emulsifier materials include glyceryl derivatives, such as glyceryl stearate. Mixtures of those materials may be used.

The compositions of the present invention may also include conventional additives which are typically used in shampoo or conditioner compositions, at their conventional art-established levels, as long as those additives are compatible with the other components and do not detract from the beneficial results delivered by the present invention. Additives which may be used in various compositions include waxes, other oils and fats and derivatives thereof, fatty acid esters of varying chain lengths, synthetic oils and fats, polymers, alcohols, polyhydric alcohols, extracts useful for providing fragrance, amino acids, nucleic acids, vitamins, hydrolyzed proteins and derivatives thereof, glycerin and derivatives thereof, enzymes, anti-inflammatory agents and other medicaments, microbiocides, anti-fungals, antiseptics, antioxidants, UV absorbers, dyes and pigments, sunscreen active agents, chelating agents, oxidizers, pH balancing agents, moisturizers and the like approved for use in formulations for human use. In addition, additives such as EDTA, glutamic acid, glycerin, panthenol, cyclomethicone, dimethicone, and pH adjustment additives may be included. Mixtures of those materials may also be used.

Examples of metal ion sequestering agents include 1-hydroxyethane-1,1-diphosphonic acid, 1-hydroxyethane-1,1-diphosphonic acid 4 Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, and trisodium hydroxyethyl ethylenediamine triacetate. Mixtures may also be used.

Examples of vitamins which may be included in the compositions of the present invention include vitamins A, B1, B2, B6, C and E and derivatives thereof; pantothenic acid and derivatives thereof; and biotin; and mixtures thereof.

Examples of antioxidants include tocopherols, dibutylhydroxytoluene, butylhydroxyanisole, and gallic acid esters. Examples of antioxidant acids include phosphoric acid, citric acid, ascorbic acid, maleic acid, malonic acid, succinic acid, lactic acid, fumaric acid, cephalin hexametaphosphate, phytic acid, and ethylenediamine tetraacetic acid. Mixtures of those materials may be used.

Various fragrances, fragrance components, and colorants may be included to provide desirable aesthetics for the compositions of the present invention.

Typical examples of germ inhibitors, which are used as preservatives acting specifically against gram-positive bacteria, include 2, 4, 4′-trichloro-2′-hydroxydiphenyl ether, chlorhexidine (1, 6-di-(4-chlorophenyl-biguano)-hexane or TCC (3, 4, 4′-trichlorocarbanilide), as well as mixtures thereof. Numerous perfumes and essential oils also have antimicrobial properties. Typical examples of such materials include eugenol, menthol and thymol in clove, mint and thyme oil.

The compositions of the present invention can be made using the following protocol: with the exception of the fragrance and preservative components, the ingredients of the present compositions are combined at a temperature of from about 70° C. to about 90° C. with adequate mixing until homogeneous. The product is then cooled, with mixing, to a temperature of less than 40° C., prior to incorporating the fragrance and preservative components. The composition is then mixed until homogeneous.

The compositions of the present invention are used to clean and condition hair in a single operation. In use, an effective amount of the composition is applied to wet hair and worked into the hair, generally using fingers of the consumer. The composition is then rinsed out of the hair and the hair is dried, either towel dried, air dried and/or using a blow dryer, generally. As used herein, the term “effective amount” is an amount of the composition of the present invention in a sufficient quantity to provide adequate cleaning and conditioning when worked into the hair, but which is not so much as to cause any undesirable effects to the hair, skin or eyes. Typically, the effective amount of the compositions of the present invention used in this defined method is from about 5 to about 30 mg. per application.

### EXAMPLES

The following examples illustrate the compositions of the present invention, as well as the use of those compositions to provide cleaning and conditioning of the hair.

| Material | #1 | #2 | #3 | #4 | #5 | #6 | #7 | #8 | #9 | #10 |
|---|---|---|---|---|---|---|---|---|---|---|
| Water | Balance | Balance | Balance | Balance. | Balance | Balance | Balance | Balance | Balance | Balance |
| pH adjuster | QS | QS | QS | QS | QS | QS | QS | QS | QS | QS |
| Cetearyl Alcohol | 6.8 | 13 | — | — | — | — | 7.5 | 13 | — | — |
| Cetyl Alcohol | — | — | 4.25 | — | 5 | — | — | — | 4.25 | — |
| Myristyl Alcohol | — | — | — | 6.7 | — | — | — | — | — | 6.7 |
| Stearyl Alcohol | — | — | — | — | 5 | — | — | — | — | — |
| Stearamidopropyl | — | — | 0.75 | — | 1 | — | — | — | 0.75 | — |
| Dimethylamine | | | | | | | | | | |
| Babassuamidopropyl | — | 3.5 | — | — | 1 | — | — | 3.5 | — | — |
| Dimethylamine | | | | | | | | | | |
| Behenamidopropyl | 3.25 | — | — | 3.25 | 1 | 3.75 | 2 | — | — | 3.25 |
| Dimethylamine | | | | | | | | | | |
| Cocamide MEA | 3 | — | — | — | — | 1.5 | 3 | — | — | — |
| Cocamide DEA | — | 5 | — | — | — | — | — | 5 | — | — |
| Lauramide MIPA | — | — | 2.5 | — | — | — | — | — | 2.5 | — |
| Soyamide DIPA | — | — | — | 1.25 | — | — | — | — | — | 1.25 |
| Capryloyl/Caproyl | — | — | — | — | 3.5 | 1.5 | — | — | — | — |
| Methyl Glucamide | | | | | | | | | | |
| Mineral Oil | — | — | — | — | — | — | — | 0.6 | — | 1 |
| Abyssinian Oil | — | — | — | — | — | — | 1 | — | — | 1 |
| Sunflower Seed Oil | — | — | — | — | — | — | — | 2 | — | — |
| Canola Oil | — | — | — | — | — | — | — | — | 5 | — |
| Fragrance | QS | QS | QS | QS | QS | QS | QS | QS | QS | QS |
| Preservative | QS | QS | QS | QS | QS | QS | QS | QS | QS | QS |

Each of the compositions above is made by combining the ingredients listed, in the order listed, at a temperature of from about 70 to about 90° C. with adequate mixing until homogeneous. The fragrance and preservative components are not included in this initial mixing or combination of ingredients. The product is then cooled, with mixing, to a temperature of less than 40° C., prior to incorporating the fragrance and preservative components. The final composition is then mixed until homogeneous.

A consumer wets his or her hair thoroughly and then applies about 15 mg. of composition #1 to the hair, working it through the hair with their fingers, thereby forming a lather in the hair. The composition (with the lather serving as a marker) is then rinsed out of the hair and the hair is first towel-dried and then dried and styled with a blower dryer. The composition both cleans and conditions the hair. Similar results are obtained when any of compositions #2-#10 are substituted in whole or in part for composition #1.

What is claimed is:

1. A conditioning shampoo composition comprising:
(a) an amidoamine having the structure: 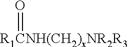
wherein R₁ is an alkyl radical with n carbon atoms (n=8-24), benzyl, and mixtures thereof; wherein R₂ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof; wherein R₃ is selected from —H, —CH₃, —CH₂OH, —CH₂CH₃, —CH₂CH₂OH, —CH₂CH₂CH₂OH, —CH₂ (CHOH)₄CH₂OH, -benzyl, and mixtures thereof; and X=1 to 3;
(b) a fatty alcohol having 12 to 24 carbon atoms;
(c) an amide surface active agent having the structure: 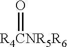
wherein R₄ is selected lauric acid, coconut acid, palmitic acid, myristic acid, behenic acid, babassu acid, isostearic acid, stearic acid, corn fatty acid, soy fatty acid, shea butter fatty acid, caprylic acid, capric acid, and mixtures thereof;
wherein R₆ is selected from —CH₂OH, —CH₂CH₂OH, —CH₂CH₂CH₂OH, —CH₂(CHOH)₄CH₂OH, -benzyl, and mixtures thereof; and
wherein R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof; and
(d) from about 5% to about 95% water;
wherein the total of components (a) and (b), is present in an amount of at least 2% by weight of the composition.

2. The composition according to claim 1 which further comprises an oil, wherein said oil is liquid at 25° C.

3. The composition according to claim 2 wherein the oil includes an oil having a chain length of at least C22 in an amount from about 50% to about 70% of the total oil component.

4. The composition according to claim 3 wherein said oil is crambe abyssinica seed oil.

5. The composition according to claim 2 wherein the oil is present in the composition at from about 0.5% to about 10%.

6. The composition according to claim 5 wherein the oil is present in the composition at from about 0.5% to about 5% of the composition.

7. The composition according to claim 1 wherein the weight ratio of component (a) to component (b) is from about 1.3:1 to about 1:7.

8. The composition according to claim 1 which further comprises a nonionic emulsifier.

9. The composition according to claim 8 wherein the emulsifier is glyceryl stearate.

10. The composition of claim 1 wherein the amidoamine is selected from stearamidopropyl dimethylamine, babassu amidopropyl dimethylamine, behenamidopropyl dimethylamine, and mixtures thereof.

11. The composition of claim 1 wherein the fatty alcohol is selected from cetearyl alcohol, cetyl alcohol, myristyl alcohol, stearyl alcohol and mixtures thereof.

12. The composition of claim 1 wherein the amide nonionic surfactant is selected from cocamide monoethanolamine, cocamide diethanolamine, lauramide monoisopropylamine, soyamide diisopropylamine, capryloyl/capryl methyl glucamide, and mixtures thereof.

13. The composition of claim 1 wherein
(a) the amidoamine is selected from stearamidopropyl dimethylamine, babassu amidopropyl dimethylamine, behenamidopropyl dimethylamine, and mixtures thereof;
(b) the fatty acid is selected from cetearyl alcohol, cetyl alcohol, myristyl alcohol, stearyl alcohol, and mixtures thereof; and
(c) the amide nonionic surfactant is selected from cocamide monoethanolamine, cocamide diethanolamine, lauramide, monoisopropylamine, soyamide diisopropylamine, capryloyl/capryl methyl glucamide, and mixtures thereof.

14. The composition of claim 1 wherein the weight ratio of component (a) to component (c) is from about 4:1 to about 1:4.

15. A method of cleaning and conditioning hair comprising the steps of thoroughly wetting the hair; applying an effective amount of the composition of claim 1 to the wet hair and working that composition through the hair; and rinsing the composition from the hair with water.

16. The method according to claim 15 wherein from about 5 to about 30 mg. of the composition is applied to the wet hair.
